(19) United States
(12) Patent Application Publication
(10) Pub. No.: US 2025/0114783 A1
(43) Pub. Date: Apr. 10, 2025

(54) SPECIMEN TREATMENT CARTRIDGE AND SHEET MEMBER

(71) Applicants: CANON MEDICAL SYSTEMS CORPORATION, Otawara-shi (JP); Canon Kabushiki Kaisha, Tokyo (JP)

(72) Inventor: Takahiro ISHII, Utsunomiya (JP)

(73) Assignees: CANON MEDICAL SYSTEMS CORPORATION, Otawara-shi (JP); Canon Kabushiki Kaisha, Tokyo (JP)

(21) Appl. No.: 18/905,856

(22) Filed: Oct. 3, 2024

(30) Foreign Application Priority Data
Oct. 6, 2023 (JP) 2023-174364

(51) Int. Cl. B01L 1/00

(52) U.S. Cl. CPC B01L 1/50

(57) ABSTRACT

A specimen treatment cartridge according to an embodiment includes a closed-system flow path, a storage container, and a sheet member. The closed-system flow path is provided for treating various types of liquids including a specimen. The storage container is a container in which the flow path is stored. The storage container includes an opening part used for accessing the flow path. The sheet member covers the opening part of the storage container. The sheet member is a member with a flexible material allowing access to the flow path in a state where the storage container is closed.

### CROSS-REFERENCE TO RELATED APPLICATIONS

This application is based upon and claims the benefit of priority from Japanese Patent Application No. 2023-174364, filed on Oct. 6, 2023, the entire contents of which are incorporated herein by reference.

### FIELD

Embodiments described herein relate generally to a specimen treatment cartridge and a sheet member.

### BACKGROUND

Examples of conventionally-known closed-system equipment include cell culture equipment constituted with a closed-system flow path and cell culture equipment constituted with a closed-system container. Such closed-system equipment may be used for treating a specimen that is infectious, in some situations. Such closed-system equipment is required to prevent entry of unwanted substances from the outside thereof.

By further storing such closed-system equipment in a container that can be closed, it may be possible to further enhance capabilities of lowering infection risks and preventing the entry of unwanted substances.

In the above-described equipment having a double closure structure, there is a possibility of encountering trouble that liquid leaks from a closed-system container to inner closed-system equipment, a tube constituting a closed-system flow path comes off, a valve controlling liquid delivery has a malfunction, or the like. In such situations, a worker (a user) needs to perform a task to resolve the trouble, after moving the equipment to an appliance such as a safety cabinet or the like while maintaining the double closure state.

However, in many cases, the appliance such as the safety cabinet is provided at a location different from where cell culturing takes place. Thus, the culture environment may be changed due to the moving of the equipment, and there is a possibility that the growth speed of the cells may be lowered or that the cells may die.

### BRIEF DESCRIPTION OF THE DRAWINGS

FIG. 1 is a perspective view illustrating an exemplary configuration of a specimen treatment cartridge according to an embodiment;

FIG. 2 is a side view illustrating the exemplary configuration of the specimen treatment cartridge according to the embodiment;

FIG. 3 is a drawing for describing an exemplary configuration of a sheet member according to the embodiment;

FIG. 4 is a drawing for describing an exemplary configuration of an outer cover according to the embodiment;

FIG. 5 is a drawing for describing an example of attaching a glove-shaped sheet and the outer cover according to the embodiment;

FIG. 6 is a drawing for describing an example of a trouble resolving task performed on closed-system equipment via the glove-shaped sheet according to the embodiment; and

FIG. 7 is a perspective view illustrating an exemplary configuration of a specimen treatment cartridge according to a second modification example.

### DETAILED DESCRIPTION

A specimen treatment cartridge according to an embodiment includes a flow path of a closed system, a storage container, and a sheet member. The flow path is provided to treat various types of liquids including a specimen. The storage container is a container in which the flow path is stored. The storage container includes an opening part used for accessing the flow path. The sheet member covers the opening part of the storage container. The sheet member is a member with a flexible material allowing access to the flow path in a state where the storage container is closed.

Exemplary embodiments of a specimen treatment cartridge and a sheet member will be described in detail below, with reference to the accompanying drawings. The specimen treatment cartridge and the sheet member of the present disclosure are not limited by the embodiments described below. In addition, as long as no conflict occurs, it is possible to combine any of the embodiments with another embodiment or conventional techniques. Also, in the following explanations, some of the constituent elements that are the same as each other may be referred to by using the same reference characters, and duplicate explanations thereof may be omitted.

A cell culture apparatus according to an embodiment of the present disclosure includes a double structure container (a cartridge) that includes a storage container in which a flow path of a closed system is stored. The cartridge is an example of the specimen treatment cartridge.

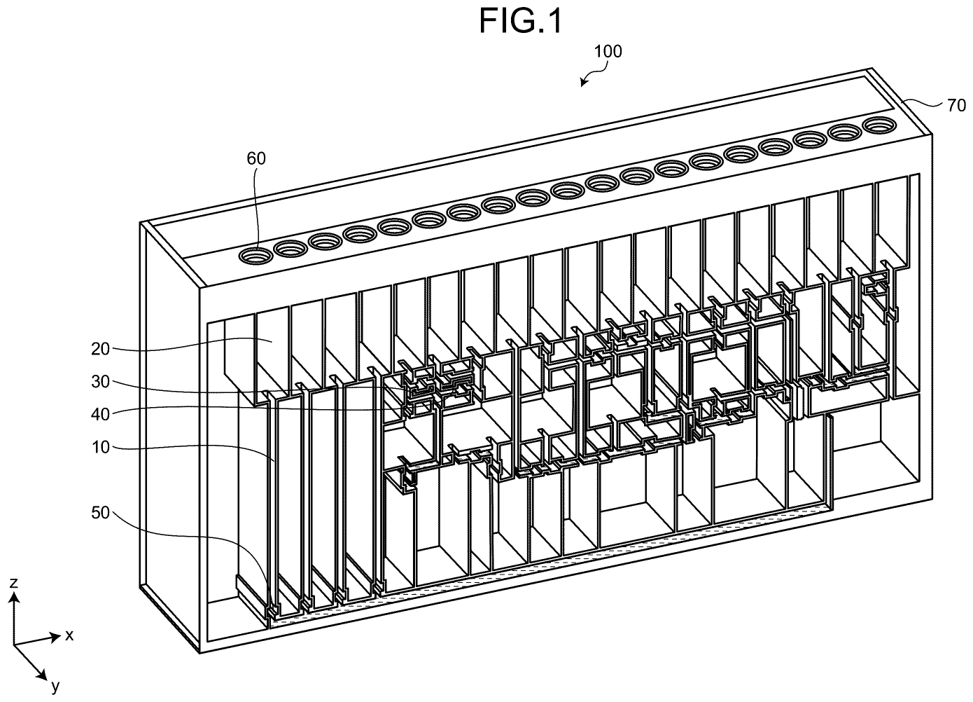

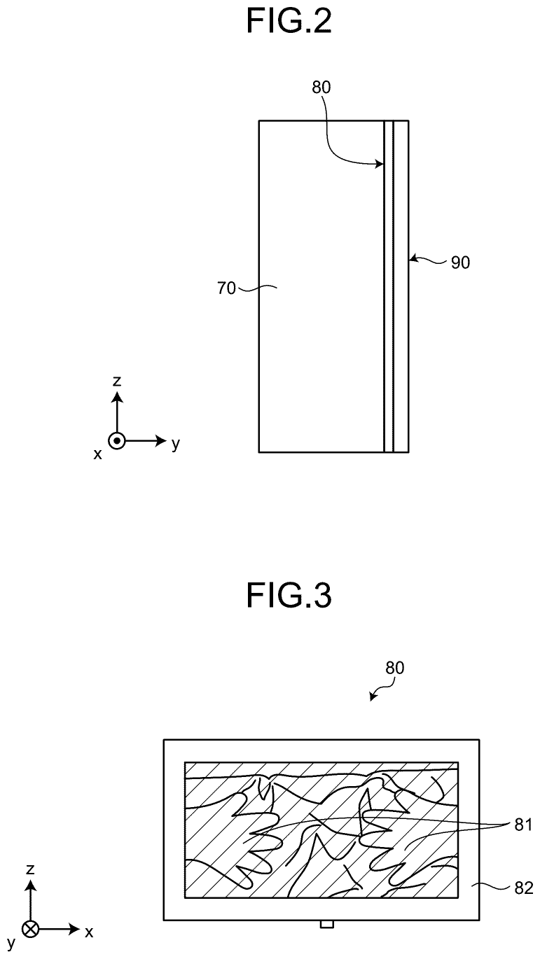

Details of the cell culture apparatus of the present embodiment will be described below, by using an example. FIG. 1 is a perspective view illustrating an exemplary configuration of a cartridge 100. FIG. 2 is a side view illustrating the exemplary configuration of the cartridge 100.

As illustrated in FIGS. 1 and 2, the cartridge 100 includes a storage container 70, a glove-shaped sheet 80, and an outer cover 90.

To begin with, a configuration stored in the storage container 70 will be described. As illustrated in FIG. 1, the storage container 70 is configured to store closed-system flow paths 10, closed-system containers 20, a filter 30, micro flow paths 40, and valves 50. The storage container 70 includes pressure increasing/reducing units 60. In the following explanations, the closed-system flow paths 10, the closed-system containers 20, the filter 30, the micro flow path 40, and the valves 50 may be referred to as closed-system equipment.

In one example, the closed-system flow paths 10 may be pipes for delivering a reagent such as a culture medium used for cell culture or a cell suspension solution or the like containing cells subject to the cell culture. The closed-system flow paths 10 are formed by using a flexible material such as silicone or resin, for example. With a pressure adjustment made by a corresponding one of the pressure increasing/reducing units 60, each of the closed-system flow paths 10 is configured to deliver the liquid such as the culture medium or the cell suspension solution in a process of the cell culture such as replacing the culture medium, from a corresponding one of the closed-system containers 20 storing the liquid to another one of the closed-system containers 20 in which the culturing is performed.

The closed-system containers 20 are containers that are used for storing various types of liquids such as biological specimens and reagents and for culturing the cells and the like. In one example, the closed-system containers 20 may be formed by using resin. The closed-system containers 20 may each be formed by using a flexible material or may each be formed with a rigid body.

The closed-system container 20 is configured to store a culture medium and deliver the stored culture medium to a corresponding one of the closed-system flow paths 10 by a pressure adjustment made by a corresponding one of the pressure increasing/reducing units 60. The closed-system container 20 is configured to store waste liquid that arises in a series of cell culturing treatment and is delivered through a corresponding one of the closed-system flow paths 10.

The filter 30 is configured to eliminate unnecessary components from a biological specimen. In one example, the filter 30 is configured to eliminate unnecessary components from the biological specimen by passing particles smaller than a predetermined size and capturing particles equal to or larger than the predetermined size.

The micro flow path 40 is configured to classify targeted cells from the biological specimen. In one example, the micro flow path 40 is configured to classify nucleated cells from blood. The micro flow path 40 may perform the classification of nucleated cells from blood by a known method, such as a method by which particles with mutually-different sizes are separated within a cross-sectional plane of the flow path by advection caused by a Dean vortex generated in the micro flow path 40 of a spiral form and a lift force dependent on a distribution of velocity on the wall surface of the flow path.

Each of the valves 50 is configured to switch between liquid delivery destinations. For example, when a closed-system container 20 storing a reagent is connected to two of the closed-system flow paths 10 via a valve 50, the valve 50 is configured to deliver the reagent to an optional one of the closed-system flow paths 10 by switching between open and closed states. With this configuration, if two of the closed-system containers 20 store mutually the same reagent, the valve 50 is able to switch between the two closed-system containers 20 to supply the reagent depending on situations.

The valves 50 are each configured to create a state in which liquid is unable to pass through a corresponding closed-system flow path 10, by pulling a magnet toward the closed-system flow path 10 and bringing the closed-system flow path 10 into a shut state. Conversely, the valves 50 are each configured to create a state in which liquid is able to pass through a corresponding closed-system flow path 10, by separating the magnet from the closed-system flow path 10 that has been in the shut state by the magnet.

Both sides of the closed-system flow path 10 are connected so as to allow communication with any of a closed-system container 20, the filter 30, the micro flow path 40, and the valve 50. The filter 30 and at least one end of the micro flow path 40 are connected to one of the closed-system flow paths 10 that is connected to either one of the closed-system container 20 and the valve 50. In addition, at least one end of each of the valves 50 is connected to one of the closed-system flow paths 10 connected to a closed-system container 20. The closed-system container 20 connected to a closed-system flow path 10 is closed so as to be out of contact with the outside.

Accordingly, the closed-system flow paths 10, the closed-system containers 20, the filter 30, the micro flow path 40, and the valves 50 as a whole constitute one closed-system flow path. In other words, it can be said that the closed-system equipment is an example of a flow path of a closed system.

An end part of the closed-system flow path 10 may be provided with a connection part (not illustrated) that can be connected so as to allow communication with another closed-system flow path 10. A so-called aseptic connection joint or the like may be used as the connection part. With this configuration, it is possible to constitute one closed-system flow path even when the configurations of the flow paths in the cartridge 100 are changed.

In FIG. 1, part of the closed-system flow paths 10 of the closed-system equipment is filled with the liquid, but possible states of the liquid present in the closed-system equipment are not limited to this example. The liquid may be present only in the closed-system containers 20. The liquid may be present in the closed-system flow paths 10 only at the time of delivering the liquid.

The pressure increasing/reducing units 60 are each configured to deliver the liquid within the closed-system equipment by increasing or reducing pressure. In one example, each of the pressure increasing/reducing units 60 is provided above a corresponding one of the closed-system containers 20 used for storing a biological specimen or a reagent. In one example, each of the pressure increasing/reducing units 60 may be connected to a pump. With the pump being driven, each of the pressure increasing/reducing units 60 delivers the liquid such as a biological specimen or a reagent stored in the corresponding closed-system container 20 to the closed-system flow path 10 connected to the closed-system container 20 by pumping the liquid out with pressure.

The storage container 70 is a container that stores the closed-system equipment. The storage container 70 has an opening. By storing the closed-system equipment in the storage container 70 and closing the opening thereof, the cartridge 100 constitutes a container that stores the biological specimens and the reagents such as a culture medium, a cell suspension solution, and/or the like, with the double structure of the closed-system equipment and the storage container 70. Alternatively, the cartridge 100 may be a multiple-layer structure container by configuring the storage container 70 to have a structure of two or more layers.

In the present embodiment, the storage container 70 is formed with a rigid body using resin, metal, or the like and has a shape capable of storing the closed-system equipment, such as a cuboid or a cube.

As illustrated in FIG. 2, the opening of the storage container 70 is covered by the glove-shaped sheet 80 and the outer cover 90. In addition, for the purpose of preventing liquid leakage of the reagents and the specimens from the storage container 70 and preventing entry of unwanted substances from the outside of the storage container 70, it is desirable to provide gaskets or the like to the parts (frames) of the glove-shaped sheet 80 and the outer cover 90 that are in contact with the storage container 70.

The storage container 70 may have a double (or multi-layer) structure in each of different sections. In one example, for culturing induced pluripotent stem (iPS) cells, a double structure may separately be provided with respect to each of the following: a section for performing delivering of a reagent (e.g., a culture medium), a section for performing a blood cell separation, a section for performing an expansion culturing, a section for performing a factor introduction, and a section for performing a planar culturing and a collecting.

Next, the glove-shaped sheet 80 will be described. The glove-shaped sheet 80 is a member configured to cover an opening part of the storage container 70 and to make it possible to access the closed-system equipment stored in the storage container 70 while the storage container 70 is in a closed state. The glove-shaped sheet 80 is an example of the sheet member.

The glove-shaped sheet 80 is formed by using a flexible material such as polyvinyl chloride (PVC). Although materials that can be used for forming the glove-shaped sheet 80 are not limited to polyvinyl chloride, it is desirable to use a material having a low reactivity to the biological specimens and the reagents used in the closed-system equipment such that various types of treatment performed by the closed-system equipment will not be affected. The glove-shaped sheet 80 may be configured to be stretchable.

The glove-shaped sheet 80 is used for a task of resolving a trouble, such as a trouble that liquid is leaking from a closed-system container 20, a trouble that one of the closed-system flow paths 10 connected to a closed-system container 20 is about to come off of the closed-system container 20, or a trouble caused by a malfunction that one of the valves 50 is unable to switch between liquid delivery destinations.

It is desirable to provide the glove-shaped sheet 80 so as to be out of contact with the closed-system equipment stored in the storage container 70 such that various types of treatment performed in the cartridge 100 will not be affected.

Next, a configuration of the glove-shaped sheet 80 will be described with reference to FIG. 3. FIG. 3 is a drawing for describing an exemplary configuration of the glove-shaped sheet 80. As illustrated in FIG. 3, the glove-shaped sheet 80 includes glove parts 81 and a frame 82.

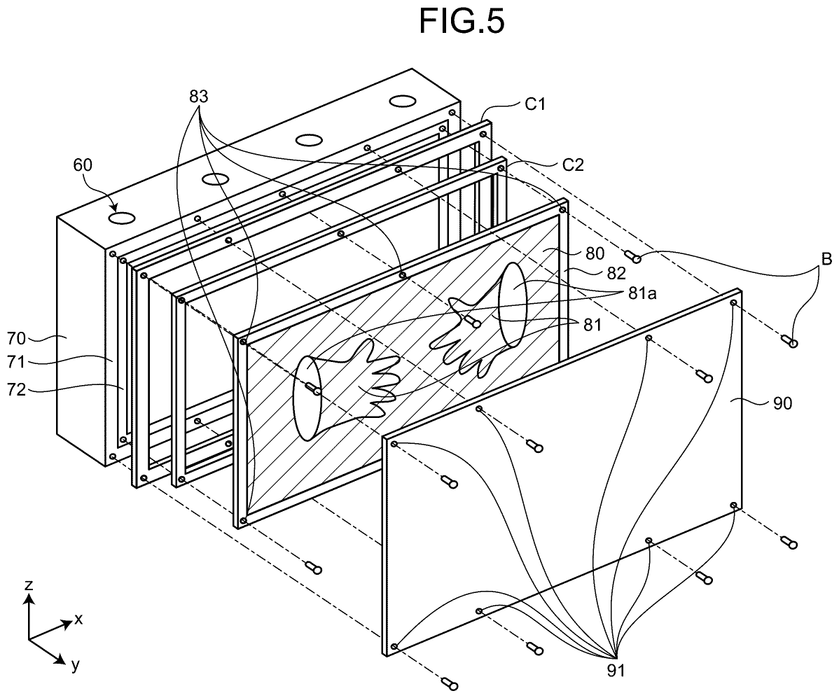

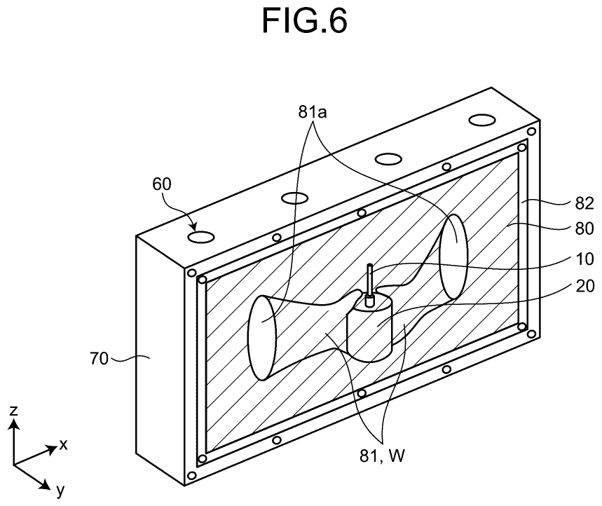

The glove parts 81 are formed in the shape of a pair of gloves to be worn on hands of a user. The glove parts 81 includes, in the outer face thereof (a face opposite to a face on the side where the storage container 70 is positioned), openings 81a (see FIGS. 5 and 6) through which the user puts his/her hands into the glove parts 81.

The user is able to access the closed-system equipment provided in the storage container 70 via the glove parts 81. The user is able to grasp the closed-system container 20 by moving his/her fingers to detach or attach any of the closed-system flow paths 10 and to manually operate the valves 50, with his/her own hands via the glove parts 81.

The frame 82 is a member for attaching the glove-shaped sheet 80 to the storage container 70. It is desirable to form the frame 82 by using a material that is harder than that of the glove-shaped sheet 80. It is acceptable to provide the frame 82 with a gasket or the like such that there is no gap between the storage container 70 and the glove-shaped sheet 80.

In the example of FIG. 3, a pair of glove parts 81 is provided on the single glove-shaped sheet 80. Alternatively, two or more pairs of glove parts 81 are provided on the single glove-shaped sheet 80. It is acceptable to provide two or more glove-shaped sheets 80 in the single storage container 70. In this case, it is also acceptable to provide a glove-shaped sheet 80 for each of different sections in the storage container 70.

Next, the outer cover 90 will be described. The outer cover 90 is a member configured to close the opening of the storage container 70, together with the glove-shaped sheet 80. In one example, the outer cover 90 is formed with a rigid body of resin or the like.

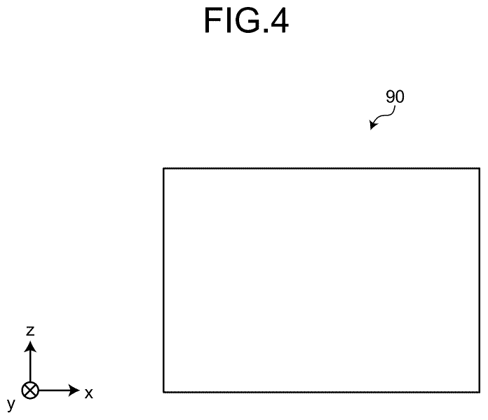

FIG. 4 is a drawing for describing an exemplary configuration of the outer cover 90. As illustrated in FIG. 4, the outer cover 90 is formed with a single plate. By attaching the outer cover 90 to the storage container 70, it is possible to prevent, with a higher probability, liquid leakage from the closed-system equipment in the storage container 70. In addition, it is possible to prevent, with a higher probability, entry of unwanted substances into the storage container 70 from the outside of the storage container 70.

Next, attachment of the glove-shaped sheet 80 and the outer cover 90 will be described with reference to FIG. 5. FIG. 5 is a drawing for describing an example of the attaching of the glove-shaped sheet 80 and the outer cover 90. FIG. 5 illustrates the example in which, while a double closure structure is formed in each of the sections inside the storage container 70, the glove-shaped sheet 80 and the outer cover 90 are to be attached to one of the sections. In FIG. 5, illustration of the closed-system equipment is omitted.

As illustrated in FIG. 5, the storage container 70 has a frame 71. The frame 71 is a member forming the opening of the storage container 70. A recessed part 72 is formed in each of the sides of the frame 71.

The outer cover 90 is formed to be larger than the glove-shaped sheet 80. A sealing member C1 is provided between the outer cover 90 and the frame 71. In addition, a sealing member C2 is provided between the glove-shaped sheet 80 and the recessed part 72 of the frame 71 of the storage container 70.

In the example of FIG. 5, eight screw fastening parts 91 are provided in an outer rim of the outer cover 90. By using the eight screw fastening parts 91, the outer cover 90 is attached to the frame 71 by using screws B so as to cover the opening of the storage container 70 and interpose the sealing member C1 between the frame 71 and the outer cover 90.

The sealing member C1 is formed by using a material such as silicone rubber or the like. The sealing member C1 is provided with through holes in which the screws B are inserted. The through holes are aligned with the positions of the eight screw fastening parts 91. The sealing member C1 has substantially the same size as the entirety of part of the frame 71 excluding the recessed part 72. The gap between the outer cover 90 and the frame 71 can be filled by interposing the sealing member C1. Therefore, it is possible to enhance hermeticity of the cartridge 100.

Moreover, six screw fastening parts 83 are provided in the frame 82 of the glove-shaped sheet 80. By using the six screw fastening parts 83, the glove-shaped sheet 80 is attached to the recessed part 72 by using screws B so as to cover the opening of the storage container 70 and interpose the sealing member C2 between the recessed part 72 and the glove-shaped sheet 80.

The sealing member C2 is formed by using a material such as silicone rubber or the like. The sealing member C2 is provided with through holes in which the screws B are inserted. The through holes are aligned with the positions of the six screw fastening parts 83. The sealing member C2 has substantially the same size as the entirety of the recessed part 72 of the frame 71. The gap between the frame 82 of the glove-shaped sheet 80 and the frame 71 can be filled by interposing the sealing member C2. Therefore, it is possible to enhance hermeticity of the cartridge 100.

The depth of the recessed part 72 is substantially equal to the sum of the thickness of the frame 82 of the glove-shaped sheet 80 and the thickness of the sealing member C2. With this configuration, there will be no protrusion when the outer cover 90 is removed from the storage container 70.

In the frame 82 of the glove-shaped sheet 80, a structure for detaching the glove-shaped sheet 80 may be provided. In one example, the frame 82 may be provided with a ring-shaped member that is supported by the frame 82 and has a diameter allowing a finger of a user to go through. In this case, after removing the screws B, the user is able to detach the glove-shaped sheet 80 from the storage container 70 by putting his/her finger in the ring-shaped member and pulling toward himself/herself.

Alternatively, it is acceptable to provide a part enabling the user to pinch by his/her fingers on a sheet part of the glove-shaped sheet 80. In this case, after removing the screws B, the user is able to detach the glove-shaped sheet 80 from the storage container 70 by pinching the part on the glove-shaped sheet 80 with his/her fingers and pulling toward himself/herself.

Next, an example of a trouble resolving task performed on the closed-system equipment via the glove-shaped sheet will be described, with reference to FIG. 6. FIG. 6 is a drawing for describing an example of the trouble resolving task performed on the closed-system equipment via the glove-shaped sheet 80. FIG. 6 depicts the example of the task performed when one of the closed-system flow paths 10 connected to a closed-system container 20 is about to come off. In FIG. 6, the user's hands except for a part thereof are omitted from the illustration.

To begin with, a user W removes the screws B of the screw fastening parts 91 in the outer cover 90 and detaches the outer cover 90. Even after the outer cover 90 is detached, the glove-shaped sheet 80 is covering the opening of the storage container 70. Thus, the closed state is maintained. Subsequently, the user W puts each of his/her hands into a different one of the two openings 81a of the glove parts 81 of the glove-shaped sheet 80.

The glove-shaped sheet 80 is formed by using a flexible material, as described above. Therefore, via the glove parts 81, the user W is able to grasp the closed-system container 20 with his/her left hand and to grasp the closed-system flow path 10 with his/her right hand. In that situation, the user W is able to, by using his/her right hand, firmly fit the closed-system flow path 10 that is about to come off into the closed-system container 20 grasped by his/her left hand.

In the present embodiment, the storage container 70 is provided with the opening only on one of the faces thereof; however, the storage container 70 may be provided with openings on both of the faces thereof. In that situation, a glove-shaped sheet 80 may be provided on each of the two faces of the storage container 70.

As described above, the cartridge 100 according to the present embodiment includes the glove-shaped sheet 80 configured to cover the opening part of the storage container 70. The glove-shaped sheet 80 is formed by using the flexible material allowing access to the closed-system equipment stored in the storage container 70 in a state where the storage container 70 is closed.

With this configuration, in the cartridge 100 according to the present embodiment, even when one of the closed-system flow paths 10 connected to a closed-system container 20 comes off due to an unsatisfactory connection, it is possible to access the closed-system flow path 10 while maintaining the closed state. Thus, it is possible to reconnect the loose closed-system flow path 10 to the closed-system container 20 while maintaining the closed state of the cartridge 100.

Accordingly, in the cartridge 100 according to the present embodiment, even when the closed-system equipment encounters trouble that may be difficult to handle from the outside of the storage container 70, the user is able to perform the operation for resolving the trouble, without moving the cartridge 100 to a safety cabinet. Thus, with the cartridge 100 according to the present embodiment, it is possible to handle the trouble having occurred on the closed-system equipment stored in the storage container 70 while maintaining the closed state of the storage container 70.

Note that it is possible to carry out any of the embodiments described above with an appropriate modification by changing part of the configurations or the functions of the apparatuses. In the following sections, modification examples of the above embodiments will be described as other embodiments. In the following sections, differences from the above embodiments will primarily be described. Detailed explanations of some of the elements that are the same as those previously described will be omitted. Additionally, the modification examples described below may be carried out individually or may be carried out as being combined with any of the above embodiments, as appropriate.

### First Modification Example

In the above-described embodiments, the glove-shaped sheet 80 is provided with the glove parts 81. However, the glove-shaped sheet 80 may be configured as a sheet without the glove parts 81.

In the present modification example, the sheet is stretchable. Thus, when the user puts his/her hands into the storage container 70 via the sheet, the sheet is deformed so as to fit the shapes of the user's hands. Accordingly, via the stretchable sheet, the user is able to access an optional position within the storage container 70.

The present modification example enables the user to access the optional position within the storage container 70, without providing the glove parts 81. Therefore, even if the storage container 70 is large in size, the stretchable single sheet makes it possible to handle trouble having occurred on the closed-system equipment stored in the storage container 70 while maintaining the closed state of the storage container 70.

### Second Modification Example

In the above embodiments, the closed-system equipment in the storage container 70 is accessed from the front face of the storage container 70, on which the opening is provided. In the present modification example, the closed-system equipment in the storage container 70 is accessed from the lateral faces (or side faces) of the storage container 70.

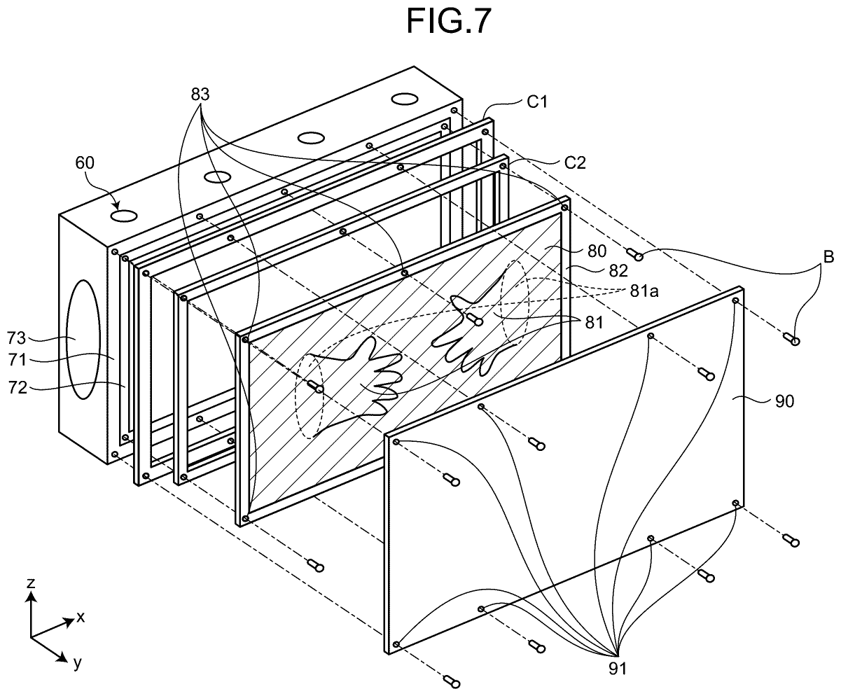

FIG. 7 is a perspective view illustrating an exemplary configuration of the cartridge 100 according to a second modification example. As illustrated in FIG. 7, the storage container 70 of the present modification example includes access openings 73 on both lateral faces. The glove-shaped sheet 80 of the present modification example is provided on an inner wall surface of the storage container 70 so as to allow communication between the openings 81a of the glove parts 81 and the access openings 73. The glove-shaped sheet 80 is attached in a manner of adhering to the inner wall surface of the storage container 70 such that no gap is formed between the access openings 73 and the glove-shaped sheet 80.

In the present modification example, the user accesses the closed-system equipment provided inside the storage container 70 through the access openings 73 provided on the lateral faces of the storage container 70. This configuration makes it possible to easily access the closed-system equipment that is located near the lateral faces of the storage container 70. In addition, by forming the outer cover 90 with a transparent material, it is possible to handle trouble having occurred on the closed-system equipment while maintaining the closed state of the storage container 70, without the need to remove the outer cover 90.

According to at least one aspect of the embodiments and the modification examples described above, it is possible for the user of the equipment having the double closure structure to handle trouble on the closed-system equipment provided inside, while maintaining the closed state of the storage container provided outside.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A specimen treatment cartridge comprising:
a flow path of a closed system for treating various types of liquids including a specimen;
a storage container in which the flow path is stored, the storage container including an opening part used for accessing the flow path; and
a sheet member covering the opening part of the storage container, the sheet member being a member with a flexible material allowing access to the flow path in a state where the storage container is closed.

2. The specimen treatment cartridge according to claim 1, wherein the sheet member includes a glove part with an opening used for accessing the flow path, the glove part having a shape of a glove to be worn on a hand of a user.

3. The specimen treatment cartridge according to claim 1, further comprising an outer cover attached to the storage container to cover an entirety of the sheet member.

4. The specimen treatment cartridge according to claim 1, wherein the flexible material of the sheet member is a stretchable material.

5. The specimen treatment cartridge according to claim 1, wherein the storage container includes access openings for accessing the flow path via the sheet member, the access openings being provided on both lateral faces of the storage container.

6. The specimen treatment cartridge according to claim 1, wherein
an interior of the storage container is segmented into multiple sections, and
the sheet member is provided for each of the sections.

7. A sheet member provided in a specimen treatment cartridge, the specimen treatment cartridge including a flow path of a closed system for treating various types of liquids including a specimen and a storage container in which the flow path is stored, the storage container including an opening part used for accessing the flow path, wherein the sheet member is configured to cover the opening part of the storage container, the sheet member being a member with a flexible material allowing access to the flow path in a state where the storage container is closed.
